(19) United States
(12) Patent Application Publication
(10) Pub. No.: US 2005/0221386 A1
(43) Pub. Date: Oct. 6, 2005

(54) Chromatographic exclusion agglutination assay and methods of use thereof

(76) Inventors: Nathan B. Turner, Gorham, ME (US); Roger N. Piasio, Cumberland, ME (US); Erik R. Piasio, Cumberland, ME (US)

Correspondence Address: T. D. FOSTER, 12760 HIGH BLUFF DRIVE, SUITE 300, SAN DIEGO, CA, 92130, US

(21) Appl. No.: 11/060,113

(22) Filed: Feb. 17, 2005

Related U.S. Application Data
(63) Continuation of application No. 60/545,419, filed on Feb. 17, 2004.

(51) Int. Cl. G01N 33/53; G01N 33/558

(52) U.S. Cl. 435/7.1; 436/514

(57) ABSTRACT

The present invention relates to agglutination assays and in particular to chromatographic exclusion assays and methods of use thereof The present invention includes a novel strategy for determining the presence of one or more analytes on interest in a test sample by using chromatographic exclusion of aggregates of bound detectable specific binding reagents that are accumulated at a particular and non-random location on the test device. In the absence of analytes of interest in the sample under test, the specific detectable binding reagent aggregates are not formed, and hence not excluded from the chromatographic media creating a distinct and readily differentiating event. The present invention is particularly adaptable as a simple test device for detection of diseases or monitoring of treatments at a doctor's office or in the home.

### FIELD OF INVENTION

The present invention relates generally to the field of agglutination assays, and particularly to chromatographic exclusion assays and methods of use thereof.

### BACKGROUND OF THE INVENTION

A variety of air agglutination assays are commercially available. These agglutination assays can be used for a variety of purposes, such as diagnosing conditions of disease or monitor treatments by analyzing trace analytes of interest in a sample. Certain agglutination assays are fully performed in solution, with the presence of visible analyte induced aggregates in the solution as indication of positive result. Other agglutination assays are performed utilizing a flow-through system where a reaction sample is passed through a small hole in order to concentrate analyte induced aggregates. Other agglutination assays are performed on dry porous strips, with the presence of anlalyte induced aggregates on the strip as indication of positive result.

Many of the available agglutination assays suffer from lack of sensitivity and are prone to providing false-positive results. The agglutination assays that utilize a dry porous strip are especially prone to providing false-positive results because of difficulties in observing and detecting true agglutinates that are aggregated in or on the porous strip from random clumping of the agglutination assay components. Most agglutination assays, especially the flow-through agglutination assays, are prone to providing false-positive results because of the presence of non-specific particulate matter in a sample under test. The above described agglutination assays usually require a highly skilled person to determine the end result accurately as positive or negative, or to make the decision to run the test again for a more definitive result. While there has been a trend to produce more sensitive agglutination assays, further improvements in the sensitivity, effectiveness, and simplicity for home or clinic use of agglutination assays are desirable, and the present invention addresses the existing problems and provides related benefits.

### SUMMARY OF THE INVENTION

The present invention relates to agglutination assays and in particular to chromatographic exclusion assays and methods of use thereof. The present invention includes a novel strategy for determining the presence of one or more analytes on interest in a test sample by using chromatographic exclusion or separation of aggregates of bound detectable specific binding reagents that are accumulated at a particular and non-random location on the test device. In the absence of analytes of interest in the sample under test, the specific detectable binding reagent aggregates are not formed, and hence not excluded from the chromatographic media creating a distinct and readily differentiating event. However, it is also possible to separate the aggregates from single particles by retaining the non-aggregated particles, as in size-exclusion chromatography. The present invention is particularly adaptable as a simple test device for detection of diseases or monitoring of treatments at a doctor's office or in the home.

The present invention recognizes that agglutination assays can be made more sensitive, more efficient, and more simple using chromatographic exclusion or separation of aggregates formed by the binding of a detectable specific binding reagent with the analytes of interest. Preferably, the chromatographically excluded or separated aggregates of a positive test result lead to a distinct event, such as the formation of a visible band on the chromatographic media, which is easily differentiated from the result of a negative test where no aggregates are excluded or separated from the chromatographic media.

A first aspect of the present invention includes a device for detecting the presence or absence of one or more analytes of interest in a test sample including a first porous member that includes one or more specific binding reagents that are capable of being detected and capable of binding to the analyte or analytes of interest suspected of being present in a test sample. The present invention also includes a second porous member that is in fluid communication with the first porous member, and is capable of substantially allowing any unbound detectable specific binding reagent to pass through freely, but substantially retaining any bound detectable specific binding reagent. When the sample is introduced to the first porous member of the present invention, the sample moves through the first and second porous member by capillary flow and the presence of the analyte or analytes of interest in the test sample is indicated by the presence of bound detectable specific binding reagent aggregates at the interface of the first porous member with the second porous member.

A second aspect of the present invention includes a device for detecting the presence or absence of one or more analytes of interest in a sample including a first porous member and a second porous member in fluid communication with one another forming a detection zone at the junction of the first and second porous members. A reaction zone positioned on the first porous member includes one or more detectable specific binding reagents that are able to bind to the analyte or analytes of interest suspected of being present in the sample under test and form aggregates. The one or more detectable specific binding reagents present at the reaction zone are capable of solublizing and moving to the second porous member by capillary flow. The second porous member is capable of substantially allowing any unbound detectable specific binding reagent to pass through freely, but substantially impeding the flow of any bound and aggregated detectable specific binding reagent. When the sample is introduced to the first porous member of the present invention, the sample moves through the first and second porous member by capillary flow and the presence of the analyte or analytes of interest in the test sample is indicated by the formation of an accumulation of bound detectable specific binding reagent aggregates at the junction of the first porous member with the second porous member, and the absence of the analyte or analytes of interest in the test sample is indicated by the absence or lack of an accumulation of bound detectable specific binding reagent aggregates at the junction of the first porous member with the second porous member.

A third aspect of the present invention includes a method of detecting the presence or absence of one or more analytes of interest in a sample including the steps of: providing a first porous member that includes one or more detectable specific binding reagents that are able to bind to the analyte or analytes of interest suspected of being present in the test sample; providing a second porous member, which is in fluid communication with the first porous member and forming a junction, such that the second porous member substantially allows any unbound detectable binding reagent to pass through freely, but substantially prevents the flow of any bound detectable specific binding reagent; introducing the test sample to the first porous member, such that the test sample moves through the first and second porous members by capillary flow and mobilizes the one or more detectable specific binding reagents of the first porous member; wherein the presence of the analyte or analytes of interest is indicated by the presence of bound detectable specific binding reagent aggregates at the junction of the first porous member with the second porous member.

A fourth aspect of the present invention includes a device for detecting the presence or absence of one or more analytes of interest in a sample including one or more detectable specific binding reagents capable of being detected and capable of binding to the one or more analytes of interest when added to the sample. The present invention also includes a first porous member and a second porous member in fluid communication with one another wherein the second porous member is capable of substantially allowing the one or more detectable specific binding reagents to pass through freely, but substantially retaining the detectable specific binding reagents when they are bound to an analyte or analytes of interest. Wherein the one or more detectable specific binding reagents are introduced to the sample forming a mixture and the mixture is then introduced to the first porous member, moving through the first and second porous members via capillary action, and the presence of the one or more analytes of interest is indicated by the presence of the bound one or more detectable specific binding reagent aggregates at the interface of the first porous member with the second porous member.

A fifth aspect of the present invention includes a method of detecting the presence or absence of one or more analytes of interest in a sample including the steps of: mixing one or more detectable specific binding reagents able to bind to said one or more analytes of interest in the sample with the sample in order to form a mixture; providing a first porous member; providing a second porous member in fluid communication with the first porous member and forming a junction. The second porous member being capable of substantially allowing the one or more detectable specific binding reagents to pass freely through the second porous member, but substantially preventing the flow of the one or more detectable specific binding reagents through the second porous member when bound to the analyte or analytes of interest; introducing the mixture to the first porous member, wherein the mixture moves by capillary action through the first and the second porous members; wherein, the presence of the one or more analytes of interest is indicated by the presence of the bound one or more detectable specific binding reagents aggregates at the junction of the first and second porous members.

A sixth aspect of the present invention includes a device for detecting the presence or absence of one or more analytes of interest in a sample including a first porous member including one or more detectable specific binding reagents capable of being detected and capable of binding to the one or more analytes of interest when added to the sample. The present invention also includes a second and a third porous members wherein the third porous member is in fluid communication with the second porous member, and the third porous member is capable of substantially allowing the one or more detectable specific binding reagents to pass freely through the third porous member, but substantially retaining the one or more detectable specific binding reagents when the one or more detectable specific binding reagents are bound to the one or more analytes of interest; whereby, the sample is introduced to the first porous member and the first porous member is brought into fluid contact with the second porous member, wherein the sample moves through the first, second, and third porous members by capillary flow and, wherein the presence of the one or more analytes of interest is indicated by the presence of the bound one or more detectable specific binding reagents aggregates at the interface of the second porous member with the third porous member.

A seventh aspect of the present invention includes a method of detecting the presence or absence of one or more analytes of interest in a sample including the steps of: providing a first porous member; providing a second porous member; providing a third porous member in fluid communication with the second porous member forming a junction, and the third porous member being capable of substantially allowing the one or more detectable specific binding reagents to pass freely through the third porous member, but substantially preventing the flow of the one or more detectable specific binding reagents through the third porous member when bound to the one or more analytes of interest; mixing one or more detectable specific binding reagents able to bind to the one or more analytes of interest in the sample with the sample forming a mixture; introducing the mixture to the first porous member; contacting the first porous member with the second porous member wherein the mixture moves by capillary action through the first, second, and third porous members; wherein, the presence of the one or more analytes of interest is indicated by the presence of the bound one or more detectable specific binding reagents aggregates at the junction of the second and third porous members.

An eighth aspect of the present invention includes a device for detecting the presence or absence of one or more analytes of interest in a test sample including one or more detectable specific binding reagents that are capable of being detected and capable of binding to the analyte or analytes of interest suspected of being present in a test sample. The present invention also includes a porous member having a first end and a second end, and is capable of substantially allowing any unbound detectable specific binding reagent to pass through freely, but substantially excluding any bound detectable specific binding reagent. When the sample is mixed with the one or more detectable binding agents and is introduced to the first end of the porous member of the present invention, the sample moves through the porous member by capillary flow and the presence of the analyte or analytes of interest in the test sample is indicated by the substantial absence of bound detectable specific binding reagent aggregates at the second end of the porous member which were excluded from the porous member at first end of the porous member.

A ninth aspect of the present invention includes method of detecting the presence or absence of one or more analytes of interest in a sample including the steps of: mixing one or more detectable specific binding reagents able to bind to said one or more analytes of interest in the sample with the sample in order to form a mixture; providing a porous member having a first end and a second end and capable of substantially allowing the one or more detectable specific binding reagents to pass freely through the porous member, but substantially preventing the flow of the one or more detectable specific binding reagents through the porous member when they are bound to the analyte or analytes of interest; introducing the mixture to the first end of the porous member, wherein the mixture moves by capillary action through the porous member; wherein, the presence of the one or more analytes of interest is indicated by the substantial absence of the bound one or more detectable specific binding reagents aggregates at the second end of the porous member that were excluded from the porous member at the first end of the porous member.

The designations “first”, “second”, or “third” as used throughout the description of the present invention are merely for purposes of convenience and are used in order to avoid any confusion. These terms are not used for and do not imply any special ordering or arrangements in terms of priority, element structure, reactivity, or the like.

### BRIEF DESCRIPTION OF THE DRAWINGS

FIG. 1A is a top view of an embodiment of the present invention.

FIG. 2B is a top view of an embodiment of the present invention.

### DETAILED DESCRIPTION OF THE INVENTION

Introduction

The present invention recognizes that agglutination assays can be made more sensitive, more efficient, and more simple using chromatographic exclusion or separation of aggregates formed by the binding of one or more detectable specific binding reagents with the analyte or analytes of interest at a particularly designated and non-random location on the test device. Preferably, the chromatographically excluded aggregates of a positive test result lead to a distinct event, such as the formation of a visible band on the chromatographic media, which is easily differentiated from the result of a negative test where no aggregates are excluded form the chromatographic media.

As a non-limiting introduction to the breadth of the present invention, the present invention includes several general and useful aspects, including:
1) A device for detecting the presence or absence of one or more analytes of interest in a test sample including:
   a first porous member that includes one or more specific binding reagents that are capable of being detected and capable of binding to the analyte or analytes of interest suspected of being present in a test sample;
   a second porous member that is in fluid communication with the first porous member. The second porous member is capable of substantially allowing any unbound detectable specific binding reagent to pass through freely, but substantially retaining any bound detectable specific binding reagent;
   wherein, the sample is introduced to the first porous member and moves though the first and second porous members by capillary flow and the presence of the analyte or analytes of interest in the test sample is indicated by the presence of bound detectable specific binding reagent aggregates at the interface of the first porous member with the second porous member.
2) A device for detecting the presence or absence of one or more analytes of interest in a sample including:
   a first porous member and a second porous member in fluid communication with one another forming a detection zone at the junction of the first and second porous members;
   a reaction zone that is positioned on the first porous member that includes one or more detectable specific binding reagent that is able to bind to the analyte or analytes of interest suspected of being present in the sample under test. The one or more detectable specific binding reagents present at the reaction zone are capable of solublizing and moving to the second porous member by capillary flow. The second porous member is capable of substantially allowing any unbound detectable specific binding reagent to pass through freely, but substantially impeding the flow of any bound detectable specific binding reagent;
   wherein, the sample is introduced to the first porous member and moves though the first and second porous members by capillary flow and the presence of the analyte of interest in the test sample is indicated by the formation of an accumulation of bound detectable specific binding reagent aggregates at the junction of the first porous member with the second porous member, and the absence of the analyte of interest in the test sample is indicated by the absence of an accumulation of bound detectable specific binding reagent aggregates at the junction of the first porous member with the second porous member.
3) A method of detecting the presence or absence of one or more analytes of interest in a sample including the steps of:
   a) providing a first porous member that includes one or more detectable specific binding reagents that are able to bind to the analyte or analytes of interest suspected of being present in the test sample;
   b) providing a second porous member, which is in fluid communication with the first porous member forming a junction, such that the second porous member substantially allows any unbound detectable binding reagent to pass through freely, but substantially prevents the flow of any bound detectable specific binding reagent;
   c) introducing the test sample to the first porous member, such that the test sample moves through the first and second porous members by capillary flow and mobilizes the one or more detectable specific binding reagent of the first porous member;
   wherein the presence of the analyte or analytes of interest is indicated by the presence of bound detectable specific binding reagent aggregates at the junction of the first porous member with the second porous member.
4) A device for detecting the presence or absence of one or more analytes of interest in a sample including:
one or more detectable specific binding reagents capable of being detected and capable of binding to the one or more analytes of interest when added to the sample;
a first porous member;
a second porous member in fluid communication with the first porous member, the second porous member being capable of substantially allowing the one or more detectable specific binding reagents to pass freely through the second porous member, but substantially retaining the one or more detectable specific binding reagents when the one or more detectable specific binding reagents are bound to the analytes of interest;
wherein, the one or more detectable specific binding reagents are introduced to the sample forming a mixture, and said mixture is introduced to said first porous member and moves through the first and second porous member by capillary flow, and the presence of the one or more analytes of interest is indicated by the presence of the bound one or more detectable specific binding reagent aggregates at the interface of the first porous member with the second porous member.
5) A method of detecting the presence or absence of one or more analytes of interest in a sample including the steps of:
a) mixing one or more detectable specific binding reagents able to bind to the one or more analytes of interest in the sample with the sample forming a mixture;
b) providing a first porous member;
c) providing a second porous member in fluid communication with the first porous member forming a junction, the second porous member being capable of substantially allowing the one or more detectable specific binding reagents to pass freely through the second porous member, but substantially preventing the flow of the one or more detectable specific binding reagents through the second porous member when bound to the one or more analytes of interest;
d) introducing the mixture to the first porous member, wherein the mixture moves by capillary action through the first and the second porous members;
wherein, the presence of the one or more analytes of interest is indicated by the presence of the bound one or more detectable specific binding reagent aggregates at the junction of the first and second porous members.
6) A device for detecting the presence or absence of one or more analytes of interest in a sample including:
a first porous member including one or more detectable specific binding reagents capable of being detected and capable of binding to the one or more analytes of interest when added to the sample;
a second porous member;
a third porous member in fluid communication with the second porous member, the third porous member being capable of substantially allowing the one or more detectable specific binding reagents to pass freely through the third porous member, but substantially retaining the one or more detectable specific binding reagents when the one or more detectable specific binding reagents are bound to the one or more analytes of interest;
whereby, the sample is introduced to the first porous member and the first porous member is brought into fluid contact with the second porous member, wherein the sample moves through the first, second, and third porous members by capillary flow and, wherein the presence of the one or more analytes of interest is indicated by the presence of the bound one or more detectable specific binding reagent aggregates at the interface of the second porous member with the third porous member.
7) A method of detecting the presence or absence of one or more analytes of interest in a sample including the steps of:
a) providing a first porous member;
b) providing a second porous member;
c) providing a third porous member in fluid communication with the second porous member forming a junction, the third porous member substantially allowing the one or more detectable specific binding reagents to pass freely through the third porous member, but substantially preventing the flow of the one or more detectable specific binding reagents through the third porous member when bound to the one or more analytes of interest;
d) mixing one or more detectable specific binding reagents able to bind to the one or more analytes of interest in the sample with the sample forming a mixture;
e) introducing the mixture to the first porous member;
f) contacting the first porous member with the second porous member wherein the mixture moves by capillary action through the first, second, and third porous members;
wherein, the presence of the one or more analytes of interest is indicated by the presence of the bound one or more detectable specific binding reagent aggregates at the junction of the second and third porous members.
8) A device for detecting the presence or absence of one or more analytes of interest in a sample including:
one or more detectable specific binding reagents capable of being detected and capable of binding to the one or more analytes of interest when added to the sample;
a porous member having a first end and a second end, the porous member being capable of substantially allowing the one or more detectable specific binding reagents to pass freely through the porous member, but substantially excluding the one or more detectable specific binding reagents when the one or more detectable specific binding reagents are bound to the analytes of interest;
wherein, the one or more detectable specific binding reagents are introduced to the sample forming a mixture and the mixture is introduced to the first end of the porous member and moves through the porous member by capillary flow and the presence of the one or more analytes of interest is indicated by the substantial absence of the bound one or more detectable specific binding reagents aggregates at the second end of the porous member that were excluded from the porous member at the first end.
9) A method of detecting the presence or absence of one or more analytes of interest in a sample including the steps of:
a) mixing one or more detectable specific binding reagents able to bind to the one or more analytes of interest in the sample with the sample forming a mixture;
b) providing a porous member having a first end and a second end being capable of substantially allowing the one or more detectable specific binding reagents to pass freely through the porous member, but substantially excluding the one or more detectable specific binding reagents through the porous member when bound to the one or more analytes of interest;
d) introducing the mixture to the first end of the porous member, wherein the mixture moves by capillary action through the porous member;
wherein, the presence of the one or more analytes of interest is indicated by the substantial absence of the bound one or more detectable specific binding reagent aggregates at the second end of the porous members that were excluded from the porous member at the first end.

The present invention relates to agglutination assays and in particular to chromatographic exclusion assays and methods of use thereof The present invention includes a novel strategy for determining the presence of one or more analytes in a test sample by using chromatographic exclusion or separation of aggregates formed by the binding of one or more detectable specific binding reagents at a defined zone of detection on the test device. The particular arrangement of the chromatographic media of the present invention is utilized in order to increase sensitivity and eliminate subjective interpretation of the test result. The present invention provides an exclusion of aggregate that is definitively detectable when the analyte or analytes of interest are present in the sample, and in the absence of the analyte or analytes of interest in the sample under test, the detectable specific binding reagent aggregates are not formed at the detection zone of the test device, and hence, are not excluded from the chromatographic media, which creates a distinct and readily differentiating event. The present invention is particularly adaptable as a simple and rapid test device for detection of diseases or monitoring of treatments at a doctor's office or in the home.

Further objectives and advantages of the present invention will become apparent as the description proceeds and when taken in conjunction with the accompanying drawings. To gain a full appreciation of the scope of the present invention, it will be further recognized that various aspects of the present invention can be combined to make desirable embodiments of the invention.

Unless defined otherwise, all technical and scientific terms used herein have the same meaning as commonly understood by one of ordinary skill in the art to which this invention belongs. Generally, the nomenclature used herein and the manufacture or laboratory procedures described below are well known and commonly employed in the art. The technical terms used herein have their ordinary meaning in the art that they are used, as exemplified by a variety of technical dictionaries. Where a term is provided in the singular, the inventor also contemplates the plural of that term. The nomenclature used herein and the procedures described below are those well known and commonly employed in the art.

Description of the Porous Members in Fluid Communication Forming a Junction

The present invention includes a first porous member, which can be made of and include any type of material, that is capable of transporting a liquid sample by capillary action, or capillary flow, wicking, or simple wetting of a liquid sample. Such material include, but are not limited to, glass fiber, nitrocellulose, paper, quartz, silicon, silica oxides, ceramics, polymeric plastics, cycloolefins, and copolymers thereof, cellulose polymers, metals, or composites made up of a combination of these materials.

An important aspect of the first porous member of the present invention is its ability to substantially allow any detectable specific binding reagent to pass freely through the first porous member, whether the detectable specific binding reagents are bound to analytes of interest or not Furthermore the pores or channels of the first porous member should be large enough as to not substantially impede or retard the free flow of any coagulated or aggregates of bound detectable specific binding reagent through the first porous member. The first porous member may have pores or channels of any size, which will be largely dependent on the size of the specific binding reagent being used or any particle or label which may be used as a label. The pore size of the first porous member may be, for example, about 10 micrometers to about 500 micrometer, and preferably about 50 micrometers to about 200 micrometers.

The present invention also includes a second porous member that is in fluid communication with the first porous member of the present invention. Preferably, one end of the first porous member is in contact and joins one end of the second porous member, forming a junction where the first porous member ends and the second porous member begins in fluid contact. Any aqueous or liquid sample that contacts the first porous member will be moved by capillary action towards the second porous member where it moves past the junction of the first and second porous members and continues to moves through the second porous member. The second porous member of the present invention can be made of and include any type of material, that is capable of transporting a liquid sample by capillary action, or capillary flow, wicking, or simple wetting of a liquid sample. Such material include, but are not limited to, glass fiber, nitrocellulose, paper, quartz, silicon, silica oxides, ceramics, polymeric plastics, cycloolefins, and copolymers thereof, cellulose polymers, metals, or composites made up of a combination of these materials.

An important aspect of the second porous member of the present invention is its ability to substantially allow any detectable specific binding reagent to pass freely through the second porous member only when the detectable specific binding reagent is not bound to any analyte. If the one or more analytes of interest are present in the test sample, binding events occur between the one or more detectable specific binding reagents of the present invention and the one or more analytes of interest of the test sample and complexes of detectable specific binding reagents and analytes are formed. Preferably, the second porous member substantially arrests or impedes the flow of any bound detectable specific binding reagent, especially when the bound detectable specific binding reagent form coagulates, agglutinates, clumps, or aggregates of detectable specific binding reagents complexes. The second porous member may have pores or channels of any size, which will be largely dependent on the size of the specific binding reagent being used or any particle or label which may be used as a label. Because the second porous member must substantially allow unbound detectable specific binding reagents to flow freely through the second porous member, the pore size of the second porous member may be, for example, about 0.1 micrometers to about 100 micrometers, and preferably about 1 micrometer to about 20 micrometers.

The capillary action provides the driving source or pumping force of the movement of the liquid through the device. The device is normally utilized in the horizontal position so that the capillary flow of the liquid sample through the device is normally a lateral flow, which is not affected by the force of gravity. However, certain embodiments of the present invention may include added driving forces, such as for example, gravity or certrifugal force.

Description of the Detectable Specific Binding Reagents of the Present Invention

The detectable specific binding reagents of the present invention are preferably substantially freely mobile when in the solublized or moist state. Preferably each type of detectable specific binding regent of the first porous member is capable of binding a single analyte of interest suspected of being present in the test sample forming aggregates, and each type of detectable specific binding reagent is distinguishable from one another. The detectable specific binding reagents can be any anti-analyte, namely a compound that is capable of specifically binding to an analyte of interest forming aggregates. The analytes of interest may include, for example, any material capable of being detected by a specific binding reagent including any cells or viruses or any components thereof. The analytes of interest may also include any small organic molecules, such as drugs, hormones, steroids, neurotransmitters, growth factors, metabolites, or other chemicals. The analytes of interest may include, for example, any large organic molecules, such as nucleic acids, proteins, polysaccharides, or other large molecules.

The detectable specific binding reagent of the present invention may be, for example, an intact antibody, such as a polyclonal or a monoclonal antibody. Preferably, the detectable specific binding regent is an antibody, for example, human or other mammalian IgG, IgG₁ IgG₂, IgG₃, IgG₄, IgM, IgA, IgY, SigA, or IgE. The detectable specific binding reagent of the present invention may also be an antibody fragment, such as an antigen-binding fragment or Fab, or an F(ab′)₂ fragment, or the antigen-binding site of an antibody (such as a complementarity-determining region of an antibody).

In some embodiments, the detectable specific binding reagent is preferably a purified, high affinity monoclonal antibody that specifically binds an analyte of interest suspected of being present in the test sample. The detectable specific binding reagent may, in other embodiments, be an antigen, a ligand, or a receptor. The detectable specific binding reagent may bind to more than one type of analyte, but preferably binds only one type of analyte that is suspected of being present in the test sample. The detectable specific binding reagent may be comprised of more than one reagent; for example, the detectable specific binding reagent may comprise more than one type of monoclonal antibody or antibody fragment, each of which specifically binds the same type of analyte of interest suspected of being present in the test sample. The detectable specific binding reagent of the present invention can include, without limitation, peptides, polypeptides, antibodies, Fab fragments, fusion proteins, chimeric or hybrid molecules, nucleic acids, nucleic acid mimics (for example, peptide nucleic acids), carbohydrates, cell surface antigens, receptors, ligands, or combinations thereof Preferably, the detectable specific binding reagent includes an antibody (monoclonal or polyclonal, natural, modified, or recombinant) or an antibody fragment (such as an Fab fragment or single-chain antibody variable region fragment). Methods of preparing, modifying, and using such antibodies or antibody fragments are known in the art (see, for example, “Antibodies: A Laboratory Manual”, E. Harlow and D. Lane, editors, Cold Spring Harbor Laboratory, 1988, 726 pp; “Monoclonal Antibodies: A Practical Approach”, P. Shepherd and C. Dean, editors, Oxford University Press, 2000, 479 pp.; and “Chicken Egg Yolk Antibodies, Production and Application: IgY-Technology (Springer Lab Manual)”, by R Schade et al., editors, Springer-Verlag, 2001, 255 pp., which are incorporated by reference in their entirety herein).

The detectable specific binding reagent can include an antigen, such as an antigen capable of specifically binding to an antibody that recognizes an analyte of interest. The detectable binding reagent can include a nucleic acid or nucleic acid mimic aptamer that binds a target such as a peptide or small molecule, or a receptor that binds a ligand, or a ligand that binds a receptor. The detectable specific binding reagent may be capable of binding to a mimotope, such as a peptide, that mimics an analyte of interest (see, for example, Kieber-Emmons (1998) Immunol. Res., 17:95-108; Shin et al. (2001) Infect. Immun., 69:3335-3342; Beenhouwer et al. (2002) J. Immunol., 169:6992-6999; Hou and Gu (2003) J. Immunol., 170:4373-4379; and Tang et al. (2003) Clin. Diagn. Lab. Immunol., 10:1078-1084, which are incorporated by reference in their entirety herein).

The detectable specific binding reagent also includes the capacity to be detected and preferably each type of detectable specific binding reagent is distinguishably detectable from one another. The detectable specific binding reagents' capacity to be detected may be any quality capable of being detected, and includes, for example, detection by a label such as a color label including dyes, metal sols, and latex particles, or capacity to be detected by a label such as, for example, fluorescent label, radioactive label, magnetic label or chemiluminescent label. The detectable specific binding reagents' capacity to be detected may also be, for example, detection by size, charge, polarity, hydrophobicity, hydrophilicity, lipophilicity, or viscosity. In order to identify one or more analytes of interest in a test sample, each detectable specific binding reagent that is specific for a certain analyte of interest may include a different capacity to be detected, for example, different color labels, different fluorescent labels, or any differentiating capacity to be distinguished, or any combination thereof.

Depending on the detectable specific binding reagents' chosen capacity to be detected, different detection methods may be employed in different protocols. Detection methods have enormous diversity and are presently available, and any detection method may be employed depending on the protocol. The detection methods may include, for example, direct detection of labels such as metal sols, colored labels, colored beads, colored particles, or fluorescent labels, either by way of direct vision or by way of a microscope. Labels of the present invention may be of any size and it will be largely dependent on the test that is to be performed and the pore size or chanel size of the porous members of the present invention. The labels of the present invention may have a size range of, for example, about 0.01 micrometers in diameter to about 50 micrometers in diameter, and preferably about 0.02 micrometers in diameter to about 2 micrometers in diameter. The detection methods may also include, for example, indirect detection of detectable labels such as magnetic labels, radioactive labels, or the measurement of scattered light to measure a change in the size population.

I. Device and Method for Detecting the Presence or Absence of One or More Analytes of Interest in a Test Sample (Internal Mixture)

The present invention includes a novel strategy for determining the presence of one or more analytes in a test sample by using chromatographic exclusion of aggregates formed by the binding of one or more detectable specific binding reagents at a defined zone of detection on the test device.

The present invention includes a first porous member including one or more detectable specific binding reagents that are each capable of being detected and each capable of biding to an analyte of interest in the test sample. The detectable specific binding reagents of the first porous members are preferably dried onto or otherwise temporarily immobilized into a reaction zone or region of the first porous member such that the reaction zone of the first porous member is impregnated by the dried detectable specific binding reagents. The impregnated detectable specific binding reagents of the first porous member are preferably substantially freely mobile when in the moist or solublized state.

An important aspect of the first porous member of the present invention is its ability to substantially allow any detectable specific binding reagent to pass freely through the first porous member, whether the detectable specific binding reagents are bound to analytes of interest or not. Furthermore the pores of the first porous member should be large enough as to not substantially impede the free flow of any coagulated or aggregates of bound detectable specific binding reagent through the first porous member.

The present invention includes a second porous member that is in fluid communication with the first porous member of the present invention. Preferably, one end of the first porous member is in contact and joins one end of the second porous member, forming a junction where the first porous member ends and the second porous member begins in fluid contact. Any aqueous or liquid sample that contacts the first porous member will be moved by capillary action towards the second porous member where it moves past the junction of the first and second porous members and continues to moves through the second porous member.

An important aspect of the second porous member of the present invention is its ability to substantially allow any detectable specific binding reagent to pass freely through the second porous member only when the detectable specific binding reagent is not bound to any analyte. Preferably, the second porous member substantially retains and impedes the flow of any bound detectable specific binding reagent, especially when the bound detectable specific binding reagent form coagulates, agglutinates, clumps, or aggregates of bound detectable specific binding reagents.

The presence or absence of one or more analytes in a test sample is determined by contacting the test sample to the first porous member of the present invention. The test sample moves through the first porous member by capillary flow where the one or more detectable specific binding reagents of the first porous member are solubilized and are mixed in with the test sample forming a mixture. If one or more analytes of interest are present in the test sample, the one or more detectable specific binding reagents bind the one or more analytes of interest forming a complex, and the detectable specific binding reagents analyte complexes move freely through the first porous member by capillary flow towards the second porous member of the present invention. At the junction of the first and second porous members, if the one or more analytes of interest are present in the test sample, the bound detectable specific binding reagents analyte complexes are substantially retained by the second porous member, which does not substantially allow bound detectable specific binding reagents analyte complexes to pass freely through the second porous member. Determining the presence of aggregated bound detectable specific binding reagents analyte complexes that are substantially accumulated at the junction of the first and second porous members indicates the presence of one or more analytes of interest in the sample. Furthermore, if the one or more detectable specific binding reagents are distinguished based on, for example, the color of different labels or the fluorescence of different labels designated for the detection and correlating to a specific analyte of interest, then the identity of each of the one or more analytes of interest suspected of being present in the test sample are revealed. Alternatively, if the one or more analytes of interest are not present in the test sample, substantially no detectable specific binding reagents analyte complexes are formed and hence are not retained by the second porous member, which allows any unbound detectable specific binding reagents to pass freely through the second porous member. Besides coagulation, agglutination, precipitation, accumulation, clumping, or plug formation, by any means or as appropriate, increasing viscosity can be used as part of the detection scheme.

In addition to the components of the present invention described above, other components may be included in the present invention, such as for example, a control zone located on the second porous member in order to ensure a test has been completed. Preferably, the control zone is located on the second porous member at some distance away from the junction of the first and second porous members. The control zone may include a region on the second porous member at some distance away from the junction of the first and second porous members, where it can indicate that the test sample has travelled past the detection zone or the junction of the first and second porous members. A control compound can be used, for example a colored solution, which can be mixed in with the test sample and be detected at the control zone indicating that the test sample has travelled through the device past the junction of the first and second porous members. The control compound may also be, for example a control detectable label such as control latex. The control zone can include immobilized specific biding reagents that are capable of detecting a control compound. The control compound, may include for example, a known substance that does not bind to the detectable specific binding regents of the first porous member. The control compound can be mixed in with the sample, which will pass through the first and second porous members without being retained at the junction of the first and second porous member and reach the control zone where it will bind to the immobilized specific binding reagent of the control zone, indicating that the test sample has travelled through the test device and has passed the junction of the first and second porous members.

In addition to the components of the present invention described above, other additives may be included in the present invention for specific purposes, such as for example, buffers, enzyme inhibitors, enzyme substrate or cofactors, preservatives, stabilizers, solublizing agents, detergents, sugars, facilitators, activators, oxidants, reductants, or any other additives desired for a particular purpose.

In addition to the components of the present invention described above, other components may be included in the present invention, such as for example, an absorbent pad in fluid communication with the first porous member of the present invention. The absorbent pad can be made of any material capable of holding an aqueous or liquid sample in fluid communication with the first porous member such that the sample can flow evenly through the first porous member by capillary action. The present invention may also include an absorbent pad in fluid communication with the distal end of the second porous member, having sufficient capacity to absorb any liquid and acting as a distal sink at the end of the test device in order to remove any excess liquid sample from the components of the present invention.

The present invention may also include, for example, a filter positioned between the first porous member and the second porous member and in a continuous fluid communication with the first and second porous members. The filter may act to filter out or trap larger components above a certain desired range, which may be present in the test sample. For example, if the test sample includes a blood sample, the filter may be used to filter out blood cells or other components of blood larger than a desired size.

The present invention is adaptable to be used with a plurality of similar first porous member and second porous member in fluid communication as described above. These plurality of porous members may be used, for example to run different desired tests on the same test sample at the same time. These plurality of porous members may be used in parallel configuration or stacked on top of one another.

In addition to the components of the present invention described above, other components may be included in the present invention, such as for example a housing. The housing may be made of any rigid material capable of housing the components of the present invention The housing may be preferably made of an impermeable material that contains the liquid sample and the components of the present invention inside the device without allowing for any leaks for permeation. The housing may be made of, for example, plastic, glass, metal, rubber, or any other non-permeable material. The housing may preferably include one or more apertures. The housing may include, for example, an aperture located on the area of the first porous member to act as an inlet for introduction of the test sample to the first porous member, located at some distance away from the junction of the first and second porous member. The housing may also include an aperture located on the area of the detection zone or the junction of the first and second porous members to allow for detection of whether the test result turned positive or negative. The housing may also include an aperture located on the area of the control zone, located on the second porous member at some distance away from the detection zone or the junction of the first and second porous members to allow for detection of whether or not the test has reached completion.

II. Device and Method for Detecting the Presence or Absence of One or More Analytes of Interest in a Test Sample (External Mixture)

The present invention includes a novel strategy for determining the presence of one or more analytes in a test sample by using chromatographic exclusion of aggregates formed by the binding of one or more detectable specific binding reagents at a defined zone of detection on the test device.

The present invention includes one or more detectable specific binding reagents that are each capable of being detected and each capable of binding to an analyte of interest in the test sample when added to the test sample. The detectable specific binding reagents of the present invention may be separately mixed with the test sample suspected of containing one or more analytes of interest forming a mixture, and incubated for a time period sufficient to allow the one or more detectable specific binding reagents to bind the one or more analytes of interest in the test sample. After proper incubation, the mixture of test sample and detectable specific binding reagents is used to run through the test device of the present invention.

The present invention includes a first porous member, which can be made of and include any type of material, that is capable of transporting a liquid sample by capillary action, or capillary flow, wicking, or simple wetting of a liquid sample.

An important aspect of the first porous member of the present invention is its ability to substantially allow any detectable specific binding reagent to pass freely through the first porous member, whether the detectable specific binding reagents are bound to analytes of interest or not. Furthermore the pores of the first porous member should be large enough as to not substantially impede the free flow of any coagulated or aggregates of bound detectable specific binding reagent through the first porous member.

The present invention includes a second porous member that is in fluid communication with the first porous member of the present invention. Preferably, one end of the first porous member is in contact and joins one end of the second porous member, forming a junction where the first porous member ends and the second porous member begins in fluid contact. Any aqueous or liquid sample that contacts the first porous member will be moved by capillary action towards the second porous member where it moves pass the junction of the first and second porous members and continues to moves through the second porous member.

An important aspect of the second porous member of the present invention is its ability to substantially allow any detectable specific binding reagent to pass freely through the second porous member only when the detectable specific binding reagent is not bound to any analyte. Preferably, the second porous member substantially retains and impedes the flow of any bound detectable specific binding reagent, especially when the bound detectable specific binding reagent form coagulates, agglutinates, clumps, or aggregates of bound detectable specific binding reagents.

A test sample may be tested by separately mixing the test sample suspected of containing one or more analytes of interest with the detectable specific binding reagents of the present invention forming a mixture, and incubated for a time period sufficient to allow the one or more detectable specific binding reagents to bind the one or more analytes of interest in the test sample. The presence or absence of one or more analytes in a test sample is determined by contacting the test sample and detectable specific binding reagent mixture to the first porous member of the present invention. If one or more analytes of interest are present in the test sample, the one or more detectable specific binding reagents bind the one or more analytes of interest forming a complex in the test mixture, and the detectable specific binding reagents analyte complexes move freely through the first porous member by capillary flow towards the second porous member of the present invention. At the junction of the first and second porous members, if the one or more analytes of interest are present in the test sample, the bound detectable specific binding reagents analyte complexes are substantially retained by the second porous member, which does not substantially allow bound detectable specific binding reagents analyte complexes to pass freely through the second porous member. Determining the presence of aggregated bound detectable specific binding reagents analyte complexes that are substantially accumulated at the junction of the first and second porous members indicates the presence of one or more analytes of interest in the sample. Furthermore, if the one or more detectable specific binding reagents are distinguished based on, for example, the color of different labels or the fluorescence of different labels designated for the detection and correlating to a specific analyte of interest, then the identity of each of the one or more analytes of interest suspected of being present in the test sample are revealed. Alternatively, if the one or more analytes of interest are not present in the test sample, substantially no detectable specific binding reagents analyte complexes are formed and hence are not retained by the second porous member, which allows any unbound detectable specific binding reagents to pass freely through the second porous member. Besides coagulation, agglutination, precipitation, accumulation, clumping, or plug formation, by any means or as appropriate, increasing viscosity can be used as part of the detection scheme.

In addition to the components of the present invention described above, other components may be included in the present invention, such as for example, a control zone located on the second porous member in order to ensure a test has been completed. Preferable, the control zone is located on the second porous member at some distance away from the junction of the first and second porous members. The control zone may include a region on the second porous member at some distance away from the junction of the first and second porous members, where it can indicate that the test sample has travelled past the detection zone or the junction of the first and second porous members. A control compound can be used, for example a colored solution, which can be mixed in with the test sample and be detected at the control zone indicating that the test sample has travelled through the device past the junction of the first and second porous members. The control compound may also be, for example a control detectable label such as control latex. The control zone can include immobilized specific biding reagents that are capable of detecting a control compound. The control compound, may include for example, a known substance that does not bind to the detectable specific binding regents used to test the test sample. The control compound can be mixed in with the sample, which will pass through the first and second porous members without being retained at the junction of the first and second porous member and reach the control zone where it will bind to the immobilized specific binding reagent of the control zone, indicating that the test sample has travelled through the test device and has passed the junction of the first and second porous members.

In addition to the components of the present invention described above, other additives may be included in the present invention for specific purposes, such as for example, buffers, enzyme inhibitors, enzyme substrate or cofactors, preservatives, stabilizers, solublizing agents, detergents, sugars, facilitators, activators, oxidants, reductants, or any other additives desired for a particular purpose.

In addition to the components of the present invention described above, other components may be included in the present invention, such as for example, an absorbent pad in fluid communication with the first porous member of the present invention. The absorbent pad can be made of any material capable of holding an aqueous or liquid sample in fluid communication with the first porous member such that the sample can flow evenly through the first porous member by capillary action. The present invention may also include an absorbent pad in fluid communication with the distal end of the second porous member, having sufficient capacity to absorb any liquid and acting as a distal sink at the end of the test device in order to remove any excess liquid sample from the components of the present invention.

The present invention may also include, for example, a filter positioned between the first porous member and the second porous member and in a continuous fluid communication with the first and second porous members. The filter may act to filter out or trap larger components above a certain desired range, which may be present in the test sample. For example, if the test sample includes a blood sample, the filter may be used to filter out blood cells or other components of blood larger than a desired size.

In addition to the components of the present invention described above, other components may be included in the present invention, such as for example a housing. The housing may be made of any rigid material capable of housing the components of the present invention The housing may be preferably made of an impermeable material that contains the liquid sample and the components of the present invention inside the device without allowing for any leaks for permeation. The housing may be made of, for example, plastic, glass, metal, rubber, or any other non-permeable material. The housing may preferably include one or more apertures. The housing may include, for example, an aperture located on the area of the first porous member to act as an inlet for introduction of the test sample to the first porous member, located at some distance away from the junction of the first and second porous member. The housing may also include an aperture located on the area of the detection zone or the junction of the first and second porous members to allow for detection of whether the test result turned positive or negative. The housing may also include an aperture located on the area of the control zone, located on the second porous member at some distance away from the detection zone or the junction of the first and second porous members to allow for detection of whether or not the test has reached completion.

III. Device and Method for Detecting the Presence or Absence of One or More Analytes of Interest in a Test Sample (Internal Mixture—3 Porous Members)

The present invention includes a novel strategy for determining the presence of one or more analytes in a test sample by using chromatographic exclusion of aggregates formed by the binding of one or more detectable specific binding reagents at a defined zone of detection on the test device. The analytes of interest may include any analyte capable of being detected.

The present invention includes a first porous member, which can be made of and include any type of material, that is capable of retaining liquid and allowing the liquid to be transported away by capillary action, or capillary flow, wicking, or simple wetting of a liquid sample. Such material include, but are not limited to, glass fiber, nitrocellulose, paper, quartz, silicon, silica oxides, ceramics, polymeric plastics, cycloolefins, and copolymers thereof, cellulose polymers, metals, or composites made up of a combination of these materials.

The present invention includes a second porous member, which can be made of and include any type of material, that is capable of transporting a liquid sample by capillary action, or capillary flow, wicking, or simple wetting of a liquid sample. Such material include glass fiber, nitrocellulose, paper, quartz, silicon, silica oxides, ceramics, polymeric plastics, cycloolefins, and copolymers thereof, cellulose polymers, metals, or composites made up of a combination of these materials.

An important aspect of the second porous member of the present invention is its ability to substantially allow any detectable specific binding reagent to pass freely through the first porous member, whether the detectable specific binding reagents are bound to analytes of interest or not. Furthermore the pores of the first porous member should be large enough as to not substantially impede the free flow of any coagulated or aggregates of bound detectable specific binding reagent through the first porous member.

The present invention includes a third porous member that is in fluid communication with the second porous member of the present invention. Preferably, one end of the second porous member is in contact and joins one end of the third porous member, forming a junction where the second porous member ends and the third porous member begins in fluid contact. Any aqueous or liquid sample that contacts the second porous member will be moved by capillary action towards the third porous member where it moves past the junction of the second and third porous members and continues to moves through the third porous member. The third porous member of the present invention can be made of and include any type of material, that is capable of transporting a liquid sample by capillary action, or capillary flow, wicking, or simple wetting of a liquid sample.

An important aspect of the third porous member of the present invention is its ability to substantially allow any detectable specific binding reagent to pass freely through the third porous member only when the detectable specific binding reagent is not bound to any analyte. Preferably, the third porous member substantially retains and impedes the flow of any bound detectable specific binding reagent, especially when the bound detectable specific binding reagent form coagulates, agglutinates, clumps, or aggregates of bound detectable specific binding reagents.

The presence or absence of one or more analytes in a test sample is determined by contacting the test sample to the first porous member of the present invention. The test sample is then retained by the first porous member where the one or more detectable specific binding reagents of the first porous member are solubilized and are mixed in with the test sample forming a mixture. If one or more analytes of interest are present in the test sample, the one or more detectable specific binding reagents bind the one or more analytes of interest forming a complex. When the first porous member of the present invention containing the test sample mixture is contacted with the second porous member of the present invention, detectable specific binding reagents analyte complexes move freely through from the first porous member to the second porous member by capillary flow towards the third porous member of the present invention. At the junction of the second and third porous members, if the one or more analytes of interest are present in the test sample, the bound detectable specific binding reagents analyte complexes are substantially retained by the third porous member, which does not substantially allow bound detectable specific binding reagents analyte complexes to pass freely through the third porous member. Determining the presence of aggregated bound detectable specific binding reagents analyte complexes that are substantially accumulated at the junction of the second and third porous members indicates the presence of one or more analytes of interest in the sample. Furthermore, if the one or more detectable specific binding reagents are distinguished based on, for example, the color of different labels or the fluorescence of different labels designated for the detection and correlating to a specific analyte of interest, then the identity of each of the one or more analytes of interest suspected of being present in the test sample are revealed. Alternatively, if the one or more analytes of interest are not present in the test sample, substantially no detectable specific binding reagents analyte complexes are formed and hence are not retained by the second porous member, which allows any unbound detectable specific binding reagents to pass freely through the second porous member. Besides coagulation, agglutination, precipitation, accumulation, clumping, or plug formation, by any means or as appropriate, increasing viscosity can be uses as part of the detection scheme.

In addition to the components of the present invention described above, other components may be included in the present invention, such as for example, a control zone located on the third porous member in order to ensure a test has been completed. Preferably, the control zone is located on the third porous member at some distance away from the junction of the second and third porous members. The control zone may include a region on the third porous member at some distance away from the junction of the second and third porous members, where it can indicate that the test sample has travelled past the detection zone or the junction of the second and third porous members. A control compound can be used, for example a colored solution, which can be mixed in with the test sample and be detected at the control zone indicating that the test sample has travelled through the device past the junction of the second and third porous members. The control compound may also be, for example a control detectable label such as control latex. The control zone can include immobilized specific biding reagents that are capable of detecting a control compound. The control compound, may include for example, a known substance that does not bind to the detectable specific binding regents of the first porous member. The control compound can be mixed in with the sample, which will pass from the first porous member to the second porous member upon contact and moves through the second and third porous members without being retained at the junction of the second and third porous member and reach the control zone where it will bind to the immobilized specific binding reagent of the control zone, indicating that the test sample has travelled through the test device and has passed the junction of the second and third porous members.

In addition to the components of the present invention described above, other additives may be included in the present invention for specific purposes, such as for example, buffers, enzyme inhibitors, enzyme substrate or cofactors, preservatives, stabilizers, detergents, sugars, facilitators, activators, oxidants, reductants, or any other additives desired for a particular purpose.

In addition to the components of the present invention described above, other components may be included in the present invention, such as for example, an absorbent pad in fluid communication with the first porous member or the second porous member of the present invention. The absorbent pad can be made of any material capable of holding an aqueous or liquid sample in fluid communication with the first porous member or the second porous member. If the absorbent pad is positioned in fluid contact with the first porous member, the sample can be retained evenly through the first porous member and be transferred evenly to the second porous member upon contact and flow evenly through the first porous member by capillary action. If the absorbent pad is positioned in fluid contact with the second porous member, the sample can be flow evenly through the second porous member and into the second porous by capillary action. The present invention may also include an absorbent pad in fluid communication with the distal end of the third porous member, having sufficient capacity to absorb any liquid and acting as a distal sink at the end of the test device in order to remove any excess liquid sample from the components of the present invention.

The present invention may also include, for example, a filter positioned between the second porous member and the third porous member and in a continuous fluid communication with the first and second porous members. The filter may act to filter out or trap larger components above a certain desired range, which may be present in the test sample. For example, if the test sample includes a blood sample, the filter may be used to filter out blood cells or other components of blood larger than a desired size.

The present invention is adaptable to be used with a plurality of similar second porous member and third porous member in fluid communication as described above. These plurality of porous members may be used, for example to run different desired tests on the same test sample at the same time. These plurality of porous members may be used in parallel configuration or stacked on top of one another.

In addition to the components of the present invention described above, other components may be included in the present invention, such as for example a housing. The housing may be made of any rigid material capable of housing the components of the present invention The housing may be preferably made of an impermeable material that contains the liquid sample and the components of the present invention inside the device without allowing for any leaks for permeation. The housing may be made of, for example, plastic, glass, metal, rubber, or any other non-permeable material. The housing may preferably include one or more apertures. The housing may include, for example, an aperture located on the area of the second porous member to act as an inlet for introduction of the test sample to the second porous member, located at some distance away from the junction of the second and third porous member. The housing may also include an aperture located on the area of the detection zone or the junction of the second and third porous members to allow for detection of whether the test result turned positive or negative. The housing may also include an aperture located on the area of the control zone, located on the third porous member at some distance away from the detection zone or the junction of the second and third porous members to allow for detection of whether or not the test has reached completion.

IV. Device and Method for Detecting the Presence or Absence of One or More Analytes of Interest in a Test Sample (External Mixture—Single Porous Member)

The present invention includes a novel strategy for determining the presence of one or more analytes in a test sample by using chromatographic exclusion of aggregates formed by the binding of one or more detectable specific binding reagents at a defined zone of detection on the test device. The analytes of interest may include any analyte capable of being detected.

The present invention includes one or more detectable specific binding reagents that are each capable of being detected and each capable of binding to an analyte of interest in the test sample when added to the test sample. The detectable specific binding reagents of the present invention may be separately mixed with the test sample suspected of containing one or more analytes of interest forming a mixture, and incubated for a time period sufficient to allow the one or more detectable specific binding reagents to bind the one or more analytes of interest in the test sample. After proper incubation, the mixture of test sample and detectable specific binding reagents is used to run through the, test device of the present invention.

The present invention includes a porous member having a first end and a second end, which can be made of and include any type of material, that is capable of transporting a liquid sample by capillary action, or capillary flow, wicking, or simple wetting of a liquid sample. Such material include glass fiber, nitrocellulose, paper, quartz, silicon, silica oxides, ceramics, polymeric plastics, cycloolefins, and copolymers thereof, cellulose polymers, metals, or composites made up of a combination of these materials.

The capillary action provides the driving source or pumping force of the movement of the liquid through the device. The device is normally utilized in the horizontal position so that the capillary flow of the liquid sample through the device is normally a lateral flow, which is not affected by the force of gravity.

An important aspect of the porous member of the present invention is its ability to substantially allow any detectable specific binding reagent to pass freely through the porous member only when the detectable specific binding reagent is not bound to any analyte. Preferably, the porous member substantially excludes any bound detectable specific binding reagent from the porous member, especially when the bound detectable specific binding reagent form coagulates, agglutinates, clumps, or aggregates of bound detectable specific binding reagents.

The capillary action provides the driving source or pumping force of the movement of the liquid through the device. The device is normally utilized in the horizontal position so that the capillary flow of the liquid sample through the device is normally a lateral flow, which is not affected by the force of gravity. However, certain embodiments of the present invention may include added driving forces, such as for example, gravity or certrifugal force.

A test sample may be tested by separately mixing the test sample suspected of containing one or more analytes of interest with the detectable specific binding reagents of the present invention forming a mixture, and incubated for a time period sufficient to allow the one or more detectable specific binding reagents to bind the one or more analytes of interest in the test sample. The presence or absence of one or more analytes in a test sample is determined by contacting the test sample and detectable specific binding reagent mixture to the first end of the porous member of the present invention. If one or more analytes of interest are present in the test sample, the one or more detectable specific binding reagents bind the one or more analytes of interest forming a complex in the test mixture, and the detectable specific binding reagents analyte complexes are substantially excluded from the porous member, which does not substantially allow bound detectable specific binding reagents analyte complexes to pass freely through the second porous member. Determining the presence of aggregated bound detectable specific binding reagents analyte complexes that are substantially accumulated at the first end of the porous members indicates the presence of one or more analytes of interest in the sample. Alternatively, detemining presence of the analyte or analytes of interest in the test sample is indicated by the substantial absence of bound detectable specific binding reagent aggregates at the second end of the porous member which were excluded from the porous member at first end of the porous member. Furthermore, if the one or more detectable specific binding reagents are distinguished based on, for example, the color of different labels or the fluorescence of different labels designated for the detection and correlating to a specific analyte of interest, then the identity of each of the one or more analytes of interest suspected of being present in the test sample are revealed. Alternatively, if the one or more analytes of interest are not present in the test sample, substantially no detectable specific binding reagents analyte complexes are formed and hence are not excluded from the porous member, which allows any unbound detectable specific binding reagents to pass freely through the porous member. Besides coagulation, agglutination, precipitation, accumulation, clumping, or plug formation, by any means or as appropriate, increasing viscosity can be uses as part of the detection scheme.

In addition to the components of the present invention described above, other additives may be included in the present invention for specific purposes, such as for example, buffers, enzyme inhibitors, enzyme substrate or cofactors, preservatives, stabilizers, detergents, sugars, facilitators, activators, oxidants, reductants, or any other additives desired for a particular purpose.

In addition to the components of the present invention described above, other components may be included in the present invention, such as for example, an absorbent pad in fluid communication with the first end, the second end, or both ends of the porous member of the present invention. The absorbent pad can be made of any material capable of holding an aqueous or liquid sample in fluid communication with the porous member such that the sample can flow evenly through the porous member by capillary action.

In addition to the components of the present invention described above, other components may be included in the present invention, such as for example a housing. The housing may be made of any rigid material capable of housing the components of the present invention The housing may be preferably made of an impermeable material that contains the liquid sample and the components of the present invention inside the device without allowing for any leaks for permeation. The housing may be made of, for example, plastic, glass, metal, rubber, or any other non-permeable material. The housing may preferably include one or more apertures. The housing may include, for example, an aperture located at the first end of the porous member to act as an inlet for introduction of the test sample to the porous member. The housing may also include an aperture located on the area of the control zone, located near the second end of the porous member to allow for detection of whether or not the test has reached completion.

### EXAMPLES

### Example I

### Detection of Staphylococcus aureus

This Example provides a device detection of methycillin resistant Staphylococcus aureaus (“MRSA”). Pastorex Staph Plus (Biorad, part #65356) was employed to demonstrate feasibility and utility of the present invention. As an experimental control, a slurry of MRSA was run on the slide agglutination assay per the manufacturer's instructions. Moderate agglutination was observed in the well containing the mixture of test latex and MRSA, and no agglutination was observed in the well containing a mixture of MRSA and the control latex.

To identify a suitable chromatographic media, the suspension of red latex test particle was applied to several types of media including nitrocellulose and porous polyethylene. A porous polyethylene membrane (Porex, part #181071) with suitable chromatographic properties was selected. When untreated particles were applied to this membrane, the pore space volume was uniformly filled with particles producing a dark red color throughout the membrane. An 18 millimeter wide ribbon of this membrane was laminated to a clear lexan support with double sided tape and cut into 6 millimeter wide test strips.

Next, 25 microliters of test particles and 25 microliters of control latex particles were dispensed into separate new tubes. To each was added 0.5 microliters of yellow food coloring to impart contrasting color to the clear solution 5 microliters of heat killed MRSA was added to each tube, mixed and allowed to incubate for 1 minute at room temperature. Twelve microliters of either test or control mixture was added to the proximal ends of separate membrane strips and the strips were rapidly filled by capillary action. As with the untreated latex above, the control latex was not excluded from the membrane and a dark red/orange color was clearly visible at the end of the strip distal to the area of sample application. However, the test latex, which had aggregated, was excluded from entering the membrane and only the yellow contrasting color of the solution was visible at the distal end of the strip.

To test the relative sensitivity of this assay system a ten-fold dilution of the MRSA slurry is tested both on the chromatographic procedure described above and by the slide agglutination assay. The diluted sample produced the same distinct yellow color indicating separation by agglutination on the chromatographic media. However, only faint and hence conclusive aggregation was observed on the slide agglutination assay. This would suggest an initial improvement in sensitivity inherent to the invention even with a single chromatographic system.

### Example II

### Detection of Streptococcus agalactiae

This Example provides a device detection of Streptococcus agalactiae (“Strep A”). Construction of the agglutination separation strip:

Materials for Construction of the Agglutination Separation Strip:

Millipore nitrocellulose: length 18 millimeter, width 6 millimeter (Millipore, Inc., Part #PK002057; 100% nitrocellulose membrane)
Bridge pads (Ahlstrom 1281; 90% cellulose fiber, 10% rayon with traces of polyacrylamide wet strength resin and polyacrylamide dry strength resin)
Absorbent pad (Ahlstrom 939; 100% cellulose with traces of polymide wet strength resin)
Lexan backing (Lexan #8010)
   Materials for Construction of the Antibody Capture Strip:
Millipore nitrocellulose: length 18 millimeter, width 6 millimeter (Millipore, Inc., Part #PK002057)
Bridge pads (Ahlstrom 1281)
Conjugate pads (Hollingsworth & Vose, H&V 7304 Nonwoven; polyester)
Lexan backing (Lexan #8010)
   Agglutination Procedure:
Remove gold conjugate pad from Configuration 1 and add to a 12×75 millimeter glass tube
Add 100 microliter of Strep A moderate control to the pad and incubate for the desired time After incubation transfer available liquid to the lower bridge pad and allow chromatography to occur for desired time
Read patient line or results at the interface where the bridge pad joins the nitrocellulose; if the analyte is present in the sample, agglutination with the gold particles will occur and deposit at the interface
   Antibody Capture Procedure:
Add 100 microliter of Strep A moderate control to the bottom bridge pad of configuration 2
Allow lateral chromatography to proceed for desired time
Read results at the antibody capture line
   Results (Experiment 1):

For non-specific binding (“NSB”), use for moderate Strep A positive control buffer

| | Agglutination | Antibody Capture |
|---|---|---|
| Visual signal intensity: | 2+ | 2 |
| NSB: | — | — |

Results (Experiment 2):

Preincubation kinetic study
Moderate Strep A positive control
NSB as in Experiment 1, above

All chromatography at 15 minutes

| Preincubation time(min) | Agglutination | Capture Antibody |
|---|---|---|
| 5 | 1 | .5 |
| 7 | 1 | .5 |
| 10 | 1 | .5 |
| 12 | 1 | .5 |
| 15 | 1 | .5 |
| 17 | 1 | .5 |
| 20 | 1 | .5 |
| NSB at 20 minutes | — | — |

### Example III

### A Detailed Embodiment of a Device ofr Detecting the Presence or Absence of One or More Analytes of Interest in a Test Sample

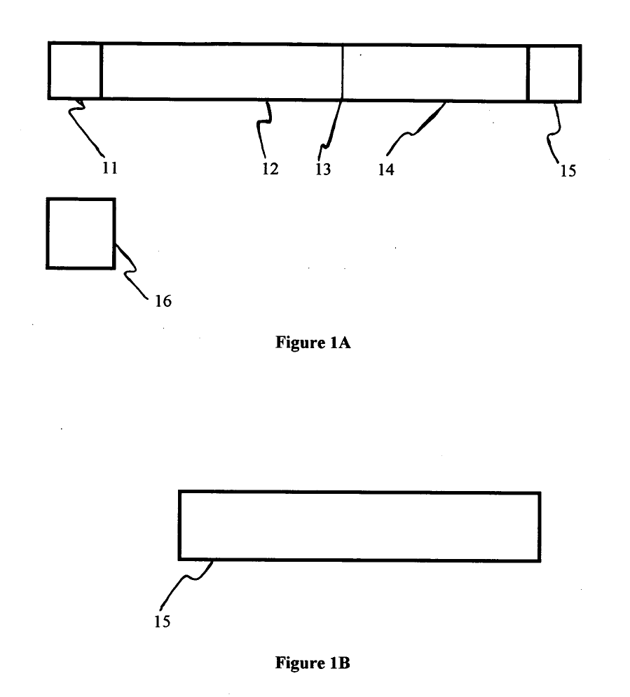

Referring to FIG. 1A the present invention includes an optional absorbent 11 pad at one end in fluid communication with the first porous member 12. The first porous member is in fluid communication with the second porous member 14, forming a junction 13. An important aspect of the first porous member 12 of the present invention is its ability to substantially allow any detectable specific binding reagent to pass freely through the first porous member 12 only when the detectable specific binding reagent is not bound to any analyte. An important aspect of the second porous member 14 is its ability to substantially arrest or impede the flow of any bound detectable specific binding reagent from the second porous member 14, especially when the bound detectable specific binding reagent form coagulates, agglutinates, clumps, or aggregates of bound detectable specific binding reagents. The present invention may optionally include an absorbent pad 14 in fluid communication with the second porous member 14. In one embodiment, the present invention may include a third porous member 16, which includes the detectable specific binding reagents and is used to contact the test device in order to run the test.

Referring to FIG. 1B the present invention includes a porous member 16, which has the ability to substantially allow any detectable specific binding reagent to pass freely through the porous member 16 only when the detectable specific binding reagent is not bound to any analyte and substantially arrest or impede the flow of any bound detectable specific binding reagent from the porous member 16, especially when the bound detectable specific binding reagent form coagulates, agglutinates, clumps, or aggregates of bound detectable specific binding reagents.

### Example IV

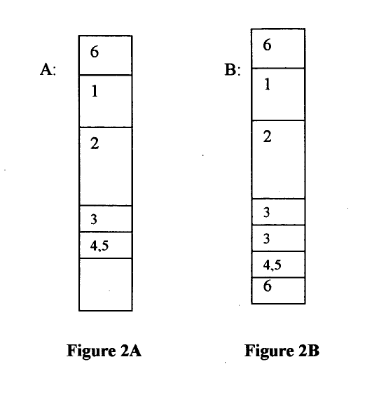

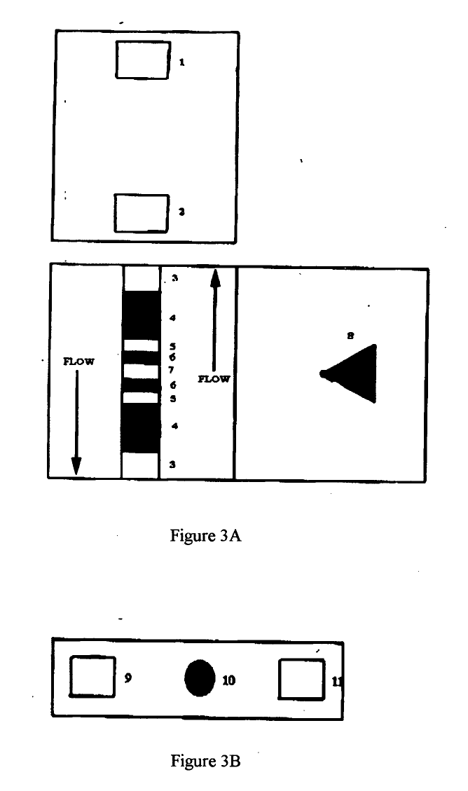

### Detection of Human Chorionic Gonadotropin (FIG. 2)

This Example refers to FIG. 2A and FIG. 2B, and provides a device detection of human chorionic gonadotropin (“hCG”).
1 means absorbent pad
2 means nitrocellulose or other membrane
3 means bridge pad
4 means conjugate pad
5 means sample pad
4,5 means pad is used both for conjugate application and sample application, either together or separately.
6 means clear plastic backing material
   Background:

The hCG (human chorionic gonadotropin) system is an excellent candidate for development of the agglutination process due to the characteristics of hormones in urine.

Hormone levels are meaningful at very low concentrations, so detection of 10-60 mIUs needs to be effective.

Procedure:

First, the test strips were constructed as shown above, and stored in a dry container.

Second, the test reagents were obtained, including:
2 vials containing hCG in urine at a concentration of 10 mIU and 60 mIU.
A sample of 440 nm latex particles adsorption coated with Goat-anti-hCG antibodies, in a 1% PEG/PBS buffer.

Third, test runs were performed by mixing the liquid latex-antibody solution mixed with the target antigen in a test tube, for zero to 10 minute incubations:
The latex/antibody was added to a test tube containing different levels (mIU) of hCG, or a negative control.
The mixture was added to one of the above agglutination devices above at the sample pad location.
In various tests, at either zero time or at a later time the sample was “pushed” with an appropriate buffer, either PBS or Malaria buffer.
The sample was then read, noting the time at which the reading was taken and the signal intensity noted, or lack of signal.
   Results:

1.—5 ul of 440 nm latex+Goat-anti-hCG-beta antibody
40 ul of 60 mIU hCG
Mix in test tube
10 minute incubation
Using agglutination device “A” above, using SR nitrocellulose for membrane (2).
Apply all 45 ul to sample application pad (4,5)
Sample was ‘pushed’ with PBS.
After 10 minutes the signal was 1+.

2.—5 ul of 440 nm latex+Goat-anti-hCG-beta antibody
40 ul of 60 mIU hCG
Mix in test tube
1 minute incubation
Using agglutination device “A” above, using SR nitrocellulose for membrane (2).
Apply all 45 ul to sample application pad (4,5)
Sample was ‘pushed’ with PBS.
After 10 minutes the signal was 0.25+

3.—3 ul of 440 nm latex+Goat-anti-hCG-beta antibody
40 ul of PBS
Mix in test tube
1 minute incubation
Using agglutination device “A” above, using SR nitrocellulose for membrane (2).
Apply all 45 ul to sample application pad (4,5)
Sample was ‘pushed’ with PBS.
After 10 minutes the signal was 0, no NSB.

4.—2 ul of 440 nm latex+Goat-anti-hCG-beta antibody
40 ul of (a) PBS, (b) 10 mIU hCG, (c) 60 mIU hCG
Mix in test tubes
10 minute incubation
Using agglutination device “A” above, using ST-type nitrocellulose for membrane (2).
Apply all 42 ul to sample application pad (4,5)
Sample was ‘pushed’ with Malaria buffer.
After 5 minutes the signal was (a) 0, no NSB, (b) 1−, and (c) 1.

5.—5 ul of 440 nm latex+Goat-anti-hCG-beta antibody
40 ul of (a) negative urine, (b) 10 mIU hCG, (c) 60 mIU hCG
Mix in test tube
2 minute incubation
Using agglutination device “A” above, using SR nitrocellulose for membrane (2).
Apply all 45 ul to sample application pad (4,5)
Sample was ‘pushed’ with Malaria buffer.
After 5 minutes the signal was (a) 0, (b) 1, (c) 1+.

6.—5 ul of 440 nm latex+Goat-anti-hCG-beta antibody
40 ul of (a) negative urine, (b) 10 mIU hCG, (c) 60 mIU hCG
Mix in test tube
3 minute incubation
Using agglutination device “A” above, using SR nitrocellulose for membrane (2).
Apply all 45 ul to sample application pad (4,5)
Sample was ‘pushed’ with Malaria buffer.
After 5 minutes the signal was (a) 0, (b) 1/1+, (c) 1/1+.

7.—5 ul of 440 nm latex+Goat-anti-hCG-beta antibody
40 ul of (a) negative urine, (b) 60 mIU hCG
Mix in test tube
No incubation
Using agglutination device “A” above, using SR nitrocellulose for membrane (2).
Apply all 45 ul to sample application pad (4,5)
Sample was ‘pushed’ with Malaria buffer.
After 5 minutes the signal was (a) 0.5+, (b) 2+/3−.

8.—440 nm latex+Goat-anti-hCG-beta antibody mixed with an equal amount of drying buffer (2% Tween-20), then dried onto the sample/conjugate pad.
40 ul of (a) negative urine, (b) 60 mIU hCG
Using agglutination device “B” above, using SR nitrocellulose for membrane (2).
Apply the 40 ul to sample/conjugate pad (4,5)
Note: there is no incubation time, this system mixes and runs instantly.
Sample was ‘pushed’ with Malaria buffer.
After 5 minutes the signal was (a) 0.5, (b) 1/1+.

### Example V

### Agglutination Deice for Dual Antigen Assay (FIG. 3)

This Example refers to FIG. 3A and FIG. 3B, and provides a device for dual antigen detection assay.

The card based assay is based on the standard ICT card assay design with an additional test strip configured in the opposite direction (FIG. 3A):
1 means window for viewing results of antigen A
2 means window for viewing results of antigen B
3 means absorbent pad (Ahlstrom 939; 100% cellulose with traces of polymide wet strength resin)
4 means Millipore nitrocellulose: length 18 millimeter, width 6 millimeter (Millipore, Inc., Part #PK002057) or Porex Corp. membrane research AB (fast flow) and research ACB (slow flow) with Porex proprietary chemical particle size exclusion barrier.
5 means bridge pad (Ahlstrom 1281; 90% cellulose fiber 10% rayon with traces of polyacrylamide wet strength resin and polyacrylamide dry strength resin)
6 means conjugate pad (Hollingsworth & Vose, H&V 7304 Nonwoven; polyester)
7 means sample pad (Ahlstrom 1281; 90% cellulose fiber, 10% rayon with traces of polyacrylamide wet strength resin and polyacrylamide dry strength resin)
8 means sample insertion port (swab based)

The cassette based assay design has the configuration of the test strips resembling that of (FIG. 3B):
9 means window for viewing results of antigen A
10 means sample port
11 means window for viewing results of antigen B
   Materials for Construction of Agglutination Dual Antigen Assay
Millipore nitrocellulose: length 18 millimeter, width 6 millimeter (Millipore, Inc., Part #PK002057: 100% nitrocellulose membrane) or Porex Corp. membrane research AB (fast flow) and research ACB (slow flow)
Bridge pads (Ahlstrom 1281; 90% cellulose fiber, 100% rayon with traces of polyacrylamide wet strength resin and polyacrylamjde dry strength resin)
Absorbent pad (Ahlstrom 939; 100% cellulose with traces of polyacrylamide wet strength. resin)
Conjugate pads (Hollingsworth & Vose, H&V 7304 Nonwoven; polyester) Lexan backing (Lexan #8010)
Sample Pad (Ahlstrom 1281; 90% cellulose fiber, 10% rayon with traces of polyacrylamide wet strength resin and polyacrylamide dry strength resin)
   Procedure

For System of FIG. 3A and System of FIG. 3B the samples are added to the sample pad in 100 microliter volumes.
If any further flow of cojugate is required, 1×PBSA (pH 7.4) is added to the sample pad.
Allow to run for 15 minutes
Depending on the system, results can either be read at the interface point between the bridge pad and nitrocellulose, or at the 300 nanometer exclusion chemical barrier applied to the Porex membrane.

The designations “first”, “second”, or “third” as used throughout this description of the present invention are merely for purposes of convenience and are used in order to avoid any confusion. These terms are not used for and do not imply any special ordering or arrangements in terms of priority, element structure, reactivity, or the like.

All headings are for the convenience of the reader and should not be used to limit the meaning of the text that follows the heading, unless so specified. Various changes and departures may be made to the present invention without departing from the spirit and scope thereof. Accordingly, it is not intended that the invention be limited to that specifically described in the specification or as illustrated in the drawings, but only as set forth in the claims.

1. A device for detecting the presence or absence of one or more analytes of interest in a sample comprising:
a first porous member including one or more detectable specific binding reagents capable of being detected and capable of binding to said one or more analytes of interest in said sample;
a second porous member in fluid communication with said first porous member, said second porous member capable of substantially allowing said one or more detectable specific binding reagents to pass freely through said second porous member, but substantially retaining said one or more detectable specific binding reagents when said one or more detectable specific binding reagents are bound to said analyte of interest;
wherein, said sample is introduced to said first porous member and moves through said first and second porous member by capillary flow and the presence of said one or more analytes of interest is indicated by the presence of said bound one or more detectable specific binding reagents aggregates at the interface of said first porous member with said second porous member.

2. The device of claim 1, further comprising an absorbent pad in fluid communication with said first porous member.

3. The device of claim 2, further comprising a filter positioned between said absorbent pad and said first porous member.

4. The device of claim 1, further comprising a control zone.

5. The device of claim 1, wherein said one or more detectable specific binding reagents comprise an antibody or an antibody fragment.

6. The device of claim 5, wherein said antibody or antibody fragment comprises a Fab fragment.

7. The device of claim 1, wherein said one or more detectable specific binding reagents comprises an antigen.

8. The device of claim 1, wherein said one or more detectable specific binding reagents comprise a receptor.

9. The device of claim 1, wherein said one or more detectable specific binding reagents each comprise a different detectable label.

10. The device of claim 9, wherein said detectable label comprises a color label.

11. The device of claim 9, wherein said detectable label comprises a fluorescent label.

12. The device of claim 9, wherein said detectable label comprises a radioactive label.

13. The device of claim 9, wherein said detectable label comprises a magnetic label.

14. The device of claim 9, wherein said detectable label comprises a chemiluminescent label.

15. A device for detecting the presence or absence of one or more analytes of interest in a sample comprising:
a first porous member and a second porous member in fluid communication with one another forming a detection zone at the junction of said first and second porous members;
a reaction zone comprising one or more detectable specific binding reagents able to bind to said one or more analytes of interest in said sample, said reaction zone positioned on said first porous member, said one or more detectable specific binding reagents capable of solublizing and moving to said second porous member by capillary flow, said second porous member substantially allowing said one or more detectable specific binding reagents to pass freely through said second porous member, but substantially impeding the flow of said one or more detectable specific binding reagents through said second porous member when bound to said one or more analytes of interest;
wherein, said sample is introduced to said first porous member and moves through said first and second porous member by capillary flow and the presence of said one or more analytes of interest in said sample is indicated by the formation of an accumulation of said bound one or more detectable specific binding reagents aggregate at the said junction, and the absence of said one or more analytes of interest in said sample is indicated by the absence of said accumulation of said bound one or more detectable specific binding reagents aggregates at the said junction.

16. The device of claim 15, further comprising an absorbent pad in fluid communication with said first porous member.

17. The device of claim 16, further comprising a filter positioned between said absorbent pad and said first porous member.

18. The device of claim 15, further comprising a control zone.

19. The device of claim 15, wherein said detectable specific binding reagent comprises an antibody or an antibody fragment.

20. The device of claim 19, wherein said antibody or antibody fragment comprises a Fab fragment.

21. The device of claim 15, wherein said one or more detectable specific binding reagents comprise an antigen.

22. The device of claim 15, wherein said one or more detectable specific binding reagents comprise a receptor.

23. The device of claim 15, wherein said one or more detectable specific binding reagents each comprise a different detectable label.

24. The device of claim 23, wherein said detectable label comprises a color label.

25. The device of claim 23, wherein said detectable label comprises a fluorescent label.

26. The device of claim 23, wherein said detectable label comprises a radioactive label.

27. The device of claim 23, wherein said detectable label comprises a magnetic label.

28. The device of claim 23, wherein said detectable label comprises a chemiluminescent label.

29. A method of detecting the presence or absence of one or more analytes of interest in a sample comprising the steps of:
a) providing a first porous member including a one or more detectable specific binding reagents able to bind to said one or more analytes of interest in said sample;
b) providing a second porous member in fluid communication with said first porous member forming a junction, said second porous member substantially allowing said one or more detectable specific binding reagents to pass freely through said second porous member, but substantially preventing the flow of said one or more detectable specific binding reagents through said second porous member when bound to said one or more analytes of interest;
c) introducing said sample to said first porous member, wherein said sample moves by capillary action through said first and said second porous members mobilizing said one or more detectable specific binding reagents of said first porous member;
wherein, the presence of said one or more analytes of interest is indicated by the presence of said bound one or more detectable specific binding reagents aggregates at said junction of said first and second porous members.

30. The method of claim 29, further comprising the step of providing an absorbent pad in fluid communication with said first porous member.

31. The method of claim 30, further comprising the step of providing a filter positioned between said absorbent pad and said first porous member.

32. The method of claims 29, further comprising a control zone to ensure completion of test.

33. The method of claim 29, wherein said one or more detectable specific binding reagents comprise an antibody or an antibody fragment.

34. The method of claim 33, wherein said antibody or antibody fragment comprises a Fab fragment.

35. The method of claim 29, wherein said one or more detectable specific binding reagents comprise an antigen.

36. The method of claim 29, wherein said one or more detectable specific binding reagents comprise a receptor.

37. The method of claim 29, wherein said one or more detectable specific binding reagents each comprise a different detectable label.

38. The method of claim 37, wherein said detectable label comprises a color label.

39. The method of claim 37, wherein said detectable label comprises a fluorescent label.

40. The method of claim 37, wherein said detectable label comprises a radioactive label.

41. The method of claim 37, wherein said detectable label comprises a magnetic label.

42. The method of claim 37, wherein said detectable label comprises a chemiluminescent label.

43. A device for detecting the presence or absence of one or more analytes of interest in a sample comprising:
one or more detectable specific binding reagents capable of being detected and capable of binding to said one or more analytes of interest when added to said sample;
a first porous member;
a second porous member in fluid communication with said first porous member, said second porous member capable of substantially allowing said one or more detectable specific binding reagents to pass freely through said second porous member, but substantially retaining said one or more detectable specific binding reagents when said one or more detectable specific binding reagents are bound to said analytes of interest;
wherein, said one or more detectable specific binding reagents are introduced to said sample forming a mixture and said mixture is introduced to said first porous member and moves through said first and second porous member by capillary flow and the presence of said one or more analytes of interest is indicated by the presence of said bound one or more detectable specific binding reagents aggregates at the interface of said first porous member with said second porous member.

44. The device of claim 43, further comprising an absorbent pad in fluid communication with said first porous member.

45. The device of claim 44, further comprising a filter positioned between said absorbent pad and said first porous member.

46. The device of claim 43, further comprising a control zone.

47. The device of claim 43, wherein said one or more detectable specific binding reagents comprise an antibody or an antibody fragment.

48. The device of claim 47, wherein said antibody or antibody fragment comprises a Fab fragment.

49. The device of claim 43, wherein said one or more detectable specific binding reagents comprises an antigen.

50. The device of claim 43, wherein said one or more detectable specific binding reagents comprise a receptor.

51. The device of claim 43, wherein said one or more detectable specific binding reagents each comprise a different detectable label.

52. The device of claim 51, wherein said detectable label comprises a color label.

53. The device of claim 51, wherein said detectable label comprises a fluorescent label.

54. The device of claim 51, wherein said detectable label comprises a radioactive label.

55. The device of claim 51, wherein said detectable label comprises a magnetic label.

56. The device of claim 51, wherein said detectable label comprises a chemiluminescent label.

57. A method of detecting the presence or absence of one or more analytes of interest in a sample comprising the steps of:
a) mixing one or more detectable specific binding reagents able to bind to said one or more analytes of interest in said sample with said sample forming a mixture;
b) providing a first porous member;
c) providing a second porous member in fluid communication with said first porous member forming a junction, said second porous member substantially allowing said one or more detectable specific binding reagents to pass freely through said second porous member, but substantially preventing the flow of said one or more detectable specific binding reagents through said second porous member when bound to said one or more analytes of interest;
d) introducing said mixture to said first porous member, wherein said mixture moves by capillary action through said first and said second porous members;
wherein, the presence of said one or more analytes of interest is indicated by the presence of said bound one or more detectable specific binding reagents aggregates at said junction of said first and second porous members.

58. The method of claim 57, further comprising the step of providing an absorbent pad in fluid communication with said first porous member.

59. The method of claim 58, further comprising the step of providing a filter positioned between said absorbent pad and said first porous member.

60. The method of claim 57, further comprising a control zone to ensure completion of test.

61. The method of claim 57, wherein said one or more detectable specific binding reagents comprise an antibody or an antibody fragment.

62. The method of claim 61, wherein said antibody or antibody fragment comprises a Fab fragment.

63. The method of claim 57, wherein said one or more detectable specific binding reagents comprise an antigen.

64. The method of claim 57, wherein said one or more detectable specific binding reagents comprise a receptor.

65. The method of claim 57, wherein said one or more detectable specific binding reagents each comprise a different detectable label.

66. The method of claim 65, wherein said detectable label comprises a color label.

67. The method of claim 65, wherein said detectable label comprises a fluorescent label.

68. The method of claim 65, wherein said detectable label comprises a radioactive label.

69. The method of claim 65, wherein said detectable label comprises a magnetic label.

70. The method of claim 65, wherein said detectable label comprises a chemiluminescent label.

71. A device for detecting the presence or absence of one or more analytes of interest in a sample comprising:
a first porous member including one or more detectable specific binding reagents capable of being detected and capable of binding to said one or more analytes of interest when added to said sample;
a second porous;
a third porous member in fluid communication with said second porous member, said third porous member being capable of substantially allowing said one or more detectable specific binding reagents to pass freely through said third porous member, but substantially retaining said one or more detectable specific binding reagents when said one or more detectable specific binding reagents are bound to said one or more analytes of interest;
whereby, said sample is introduced to said first porous member and said first porous member is brought into fluid contact with said second porous member, wherein said sample moves through said first, second, and third porous members by capillary flow and, wherein the presence of said one or more analytes of interest is indicated by the presence of said bound one or more detectable specific binding reagents aggregates at the interface of said second porous member with said third porous member.

72. The device of claim 71, further comprising an absorbent pad in fluid communication with said second porous member.

73. The device of claim 72, further comprising a filter positioned between said absorbent pad and said second porous member.

74. The device of claim 71, further comprising a control zone.

75. The device of claim 71, wherein said one or more detectable specific binding reagents comprise an antibody or an antibody fragment.

76. The device of claim 75, wherein said antibody or antibody fragment comprises a Fab fragment.

77. The device of claim 71, wherein said one or more detectable specific binding reagents comprises an antigen.

78. The device of claim 71, wherein said one or more detectable specific binding reagents comprise a receptor.

79. The device of claim 71, wherein said one or more detectable specific binding reagents each comprise a different detectable label.

80. The device of claim 79, wherein said detectable label comprises a color label.

81. The device of claim 79, wherein said detectable label comprises a fluorescent label.

82. The device of claim 79, wherein said detectable label comprises a radioactive label.

83. The device of claim 79, wherein said detectable label comprises a magnetic label.

84. The device of claim 79, wherein said detectable label comprises a chemiluminescent label.

85. A method of detecting the presence or absence of one or more analytes of interest in a sample comprising the steps of:
a) providing a first porous member;
b) providing a second porous member;
c) providing a third porous member in fluid communication with said second porous member forming a junction, said third porous member substantially allowing said one or more detectable specific binding reagents to pass freely through said third porous member, but substantially preventing the flow of said one or more detectable specific binding reagents through said third porous member when bound to said one or more analytes of interest;
d) mixing one or more detectable specific binding reagents able to bind to said one or more analytes of interest in said sample with said sample forming a mixture;
e) introducing said mixture to said first porous member;
f) contacting said first porous member with said second porous member wherein said mixture moves by capillary action through said first, second, and third porous members;
wherein, the presence of said one or more analytes of interest is indicated by the presence of said bound one or more detectable specific binding reagents aggregates at said junction of said second and third porous members.

86. The method of claim 85, further comprising the step of providing an absorbent pad in fluid communication with said first porous member.

87. The method of claim 86, further comprising the step of providing a filter positioned between said absorbent pad and said first porous member.

88. The method of claim 85, further comprising a control zone to ensure completion of the test.

89. The method of claim 85, wherein said one or more detectable specific binding reagents comprise an antibody or an antibody fragment.

90. The method of claim 89, wherein said antibody or antibody fragment comprises a Fab fragment.

91. The method of claim 85, wherein said one or more detectable specific binding reagents comprise an antigen.

92. The method of claim 85, wherein said one or more detectable specific binding reagents comprise a receptor.

93. The method of claim 85, wherein said one or more detectable specific binding reagents each comprise a different detectable label.

94. The method of claim 93, wherein said detectable label comprises a color label.

96. The method of claim 93, wherein said detectable label comprises a fluorescent label.

97. The method of claim 93, wherein said detectable label comprises a radioactive label.

98. The method of claim 93, wherein said detectable label comprises a magnetic label.

99. The method of claim 93, wherein said detectable label comprises a chemiluminescent label.

100. A device for detecting the presence or absence of one or more analytes of interest in a sample comprising:
one or more detectable specific binding reagents capable of being detected and capable of binding to said one or more analytes of interest when added to said sample;
a porous member having a first end and an second end, said porous member capable of substantially allowing said one or more detectable specific binding reagents to pass freely through said porous member, but substantially excluding said one or more detectable specific binding reagents when said one or more detectable specific binding reagents are bound to said analytes of interest;
wherein, said one or more detectable specific binding reagents are introduced to said sample forming a mixture and said mixture is introduced to said first end of the porous member and moves through said porous member by capillary flow and the presence of said one or more analytes of interest is indicated by the substantial absence of said bound one or more detectable specific binding reagents aggregates at said second end of said porous member that were excluded form said porous member at said first end.

101. The device of claim 100, further comprising an absorbent pad in fluid communication with said first porous member.

102. The device of claim 101, further comprising a filter positioned between said absorbent pad and said first porous member.

103. The device of claim 100, further comprising a control zone.

104. The device of claim 100, wherein said one or more detectable specific binding reagents comprise an antibody or an antibody fragment.

105. The device of claim 104, wherein said antibody or antibody fragment comprises a Fab fragment.

106. The device of claim 100, wherein said one or more detectable specific binding reagents comprises an antigen.

107. The device of claim 100, wherein said one or more detectable specific binding reagents comprise a receptor.

108. The device of claim 100, wherein said one or more detectable specific binding reagents each comprise a different detectable label.

109. The device of claim 108, wherein said detectable label comprises a color label.

110. The device of claim 108, wherein said detectable label comprises a fluorescent label.

111. The device of claim 108, wherein said detectable label comprises a radioactive label.

112. The device of claim 108, wherein said detectable label comprises a magnetic label.

113. The device of claim 108, wherein said detectable label comprises a chemiluminescent label.

114. A method of detecting the presence or absence of one or more analytes of interest in a sample comprising the steps of:
a) mixing one or more detectable specific binding reagents able to bind to said one or more analytes of interest in said sample with said sample forming a mixture;
c) providing a porous member having a first end and a second end, said porous member being capable of substantially allowing said one or more detectable specific binding reagents to pass freely through said porous member, but substantially excluding said one or more detectable specific binding reagents from said porous member when bound to said one or more analytes of interest;
d) introducing said mixture to said first end of said porous member, wherein said mixture moves by capillary action through said porous member;
wherein, the presence of said one or more analytes of interest is indicated by the substantial absence of said bound one or more detectable specific binding reagents aggregates at said second end of said porous member that were excluded from said porous member at said first end.

115. The method of claim 114, further comprising the step of providing an absorbent pad in fluid communication with said first porous member.

116. The method of claim 115, further comprising the step of providing a filter positioned between said absorbent pad and said first porous member.

117. The method of claim 114, further comprising a control zone to ensure completion of test.

118. The method of claim 114, wherein said one or more detectable specific binding reagents comprise an antibody or an antibody fragment.

119. The method of claim 118, wherein said antibody or antibody fragment comprises a Fab fragment.

120. The method of claim 114, wherein said one or more detectable specific binding reagents comprise an antigen.

121. The method of claim 114, wherein said one or more detectable specific binding reagents comprise a receptor.

122. The method of claim 114, wherein said one or more detectable specific binding reagents each comprise a different detectable label.

123. The method of claim 122, wherein said detectable label comprises a color label.

124. The method of claim 122, wherein said detectable label comprises a fluorescent label.

125. The method of claim 122, wherein said detectable label comprises a radioactive label.

126. The method of claim 122, wherein said detectable label comprises a magnetic label.

127. The method of claim 122, wherein said detectable label comprises a chemiluminescent label.
